(19) United States
(12) Patent Application Publication
(10) Pub. No.: US 2006/0088615 A1
(43) Pub. Date: Apr. 27, 2006

(54) Compositions containing Malva sylvestris extract and use thereof on skin and mucosal tissues

(76) Inventors: Miri Seiberg, Princeton, NJ (US); Violetta Iotsova Stone, Robbinsville, NJ (US); Renbin Zhao, Plainsboro, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/973,314

(22) Filed: Oct. 26, 2004

(51) Int. Cl. A61K 36/185

(52) U.S. Cl. 424/769

(57) ABSTRACT

The present invention relates to a method of enhancing the elasticity or structural integrity of skin or mucosal tissue by administering to skin or mucosal tissue in need of such enhancement a composition containing a safe and effective amount of Malva sylvestris extract.

### BACKGROUND OF THE INVENTION

Aging of the skin is a complex phenomenon resulting from the interaction of several intrinsic and extrinsic factors. Intrinsic aging is an inevitable, genetically programmed process. Among extrinsic influences (e.g., wind, heat, cigarette smoke, chemicals, etc.), ultraviolet radiation appears to be the single most important factor associated with aging of the skin. The effect of ultraviolet radiation on elastic tissues results in elastosis, which is the accumulation of damaged elastin, resulting in reduced elasticity and resilience.

Elastin is a critical component of extracellular matrix, and is especially abundant in tissues subject to physical deformations, such as lungs, blood vessels and skin.

The effect of intrinsic aging on tissue elasticity of mucosal tissues (such as vaginal, oral, or rectal mucosal tissues) and of viscero-elastic tissues (that are lining body cavities such as the respiratory track, the gastro-intestinal track, the urinal and bladder track, or the reproductive track) is very similar to the effect of intrinsic skin aging. Elastin fiber production in these tissues is reduced with aging, resulting in reduced responsiveness to stimuli. In the oral cavity, such changes can contribute to a decrease in the health of the gums (leading to reduced resistance to the pressure of food processing), increased gum bleeding, loose teeth, and a general decrease in the visual health parameters of the oral cavity. In the vagina, reduced elastin fiber production could result in stiffness and reduced sexual function, and uterine prolapse is associated with reduced elasticity of the female reproductive system. Reduced elasticity of the bladder can result in urine incontinence. In the eye, degenerative changes in elastin fibers in Brunch's membrane can be responsible for deposition of drusen and macular degeneration. Consequently, the reduction in elasticity of these tissues results in reduced quality of life and self esteem.

Thus, it is desired to have a treatment that can prevent, retard, or reverse the intrinsic aging effects on tissue elasticity.

Malvaceae is a family of flowering plants that includes the mallows, cotton plants, okra plants, hibiscus, baobab trees, and balsa trees. The family traditionally consists of about 1,500 species in 75 genera. Malva sylvestris is a species from the Malva (mallow) genera. The leaves of Malva sylvestris, otherwise known as blue mallow, are rich in mucilage. The mucilage of M. sylvestris is made up of high molecular weight acidic polysaccharides (Classen B, et al., Planta Med 64(7): 640-44 (1988)). The leaf tea is traditionally believed to be useful as an anti-inflammatory, decongestant, humectant, expectorant, and laxative. It has also been used internally for soothing sore throats, laryngitis, tonsillitis, coughs, dryness of the lungs, and digestive upsets. Mallow is also used as a poultice for healing wounds and skin inflammations. In traditional medicine, mallow leaf tea is also used against abnormal growths of the stomach and to alleviate urinary infections (Bisset N G (ed). Malvae folium—Mallow leaf. In Herbal Drugs and Phyto-pharmaceuticals (1994, CRC Press, Stuttgart, pp 313-316). Studies on irritated mucous membranes have shown that the mucilage of Malva sylvestris binds to buccal membranes and other mucous membranes of the body (Schmidgall J, et al. Planta Med 66(1): 48-53(2000)).

Cotinus coggygria extract is traditionally believed to be useful as an anti-microbial treatment, used in the form of external washes. See, e.g., U.S. Patent Applications No. 2002/0132021 where the extract is mentioned to be active against E. coli, Staphylococcus aureus and S. cerevisiae, as well as having anti-cancer activity. The dried leaf and twig of Cotinus coggygria is used in Chinese traditional medicine to eliminate “dampness” and “heat”, and as an antipyretic (Huang K. C., The Pharmacology of Chinese Herbs (CRS Press, 1999, pp 193-194). A yellow/orange dye can be obtained from the root and stem and can be used for fabric dying. The leaves and bark are a good source of tannins (Grieve M. A Modern Herbal. Dover Publications, Inc. NY, 1971, pp 779-781).

The present invention relates to the unexpected discovery that Malva sylvestris and Cotinus coggygria extracts are both effective for enhancing the elasticity of the skin and mucosal tissues.

### SUMMARY OF THE INVENTION

In one aspect, the present invention relates to a method of enhancing the elasticity or structural integrity of skin or mucosal tissue by administering to skin or mucosal tissue in need of such enhancement a composition containing a safe and effective amount of Malva sylvestris extract.

In another aspect, the present invention features a product including a composition comprising a Malva sylvestris extract and instructions directing the user to apply the composition to the skin or mucosal tissue in order to enhance the elasticity or structural integrity of such skin or mucosal tissue.

In another aspect, the present invention features a method of promoting a product including a composition containing a Malva sylvestris extract by directing the user to apply said composition to skin or mucosal tissue to enhance the elasticity or structure integrity of the skin or mucosal tissue.

Other features and advantages of the present invention will be apparent from the detailed description of the invention and from the claims.

### DETAILED DESCRIPTION OF THE INVENTION

It is believed that one skilled in the art can, based upon the description herein, utilize the present invention to its fullest extent. The following specific embodiments are to be construed as merely illustrative, and not limitative of the remainder of the disclosure in any way whatsoever.

Unless defined otherwise, all technical and scientific terms used herein have the same meaning as commonly understood by one of ordinary skill in the art to which the invention belongs. Also, all publications, patent applications, patents, and other references mentioned herein are incorporated by reference. Unless otherwise indicated, a percentage refers to a percentage by weight (i.e., % (W/W)).

Definitions

What is meant by “enhancing the elasticity or structural integrity” is increasing, preventing the loss, or retarding the loss of elasticity or structural integrity of the tissue, including but not limited to, treating sagging, lax and loose tissue, tightening skin or mucosal tissues. The loss of elasticity or tissue structure integrity may be a result of a number of factors, including but not limited to disease, aging, hormonal changes, mechanical trauma, environmental damage, or the result of an application of products, such as a cosmetics or pharmaceuticals, to the tissue.

What is meant by “mucosal tissues” are tissues that express elastin and are composed in part of cells of mesenchymal and epithelial origin. Examples of mucosal tissues include, but are not limited to, vaginal, oral, corneal, rectal, and viscero-elastic tissues. Examples of viscero-elastic tissues are those that line the respiratory track, blood vessel walls, the gastro-intestinal track, the urinal and bladder track, and the reproductive track.

What is meant by a “product” is a product in finished packaged form. In one embodiment, the package is a container such as a plastic, metal or glass tube or jar containing the composition. The product may further contain additional packaging such as a plastic or cardboard box for storing such container. In one embodiment, the product contains instructions directing the user to administer the composition to the tissue to enhance its elasticity. Such instructions may be printed on the container, label insert, or on any additional packaging.

What is meant by “promoting” is promoting, advertising, or marketing. Examples of promoting include, but are not limited to, written, visual, or verbal statements made on the product or in stores, magazines, newspaper, radio, television, internet, and the like. Examples of such statements include, but are not limited to, “enhances skin elasticity or structural integrity,” “improving visible and tactilely perceptible manifestations of the skin,” “increases skin elasticity or structure,” “restores skin elasticity or structure,” “treats sagging or lax skin,” “enhances vaginal elasticity,” “enhances sexual satisfaction,” “increases vaginal elasticity,” “restores vaginal elasticity,” “strengthen vaginal wall,” “treats vaginal prolapse,” “enhances gum elasticity,” “increases gum elasticity,” “restores gum elasticity,” “enhances alveolar wall elasticity,” “increases alveolar wall elasticity,” and “restores alveolar wall elasticity.”

As used herein, “administering” means contacting the tissue, e.g., by use of the hands or an applicator such, but not limited to, a wipe, tube, roller, spray, vaginal applicator, patch, tampon, toothbrush, suppository, inhaler, nasal spray, nasal dropper, eye dropper, contact lens, candy, and gums.

As used herein, “composition” means a composition suitable for administration to the skin or mucosal tissue.

As used herein, “cosmetically-acceptable” means that the ingredients which the term describes are suitable for use in contact with tissues (e.g., the skin or hair, vulval, vaginal, nasal, laryngeal, tracheal, eye or buccal tissue) without undue toxicity, incompatibility, instability, irritation, allergic response, and the like.

As used herein, “safe and effective amount” means an amount of the extract or of the composition sufficient to induce an enhancement in tissue elasticity, but low enough to avoid serious side effects. The safe and effective amount of the compounds or composition will vary with the area being treated, the age, health and skin type of the end user, the duration and nature of the treatment, the specific extract, ingredient, or composition employed, the particular cosmetically-acceptable carrier utilized, and like factors.

Malva Sylvestris Extract

What is meant by a “Malva sylvestris extract” is a blend of compounds isolated from the plant Malva sylvestris. In one embodiment, the compounds are isolated from the flowers of the plant. In a further embodiment, the compounds are isolated from dried flowers of the plant. Such compounds may be isolated from one or more part of the plant (e.g., the whole plant, flower, seed, root, rhizome, stem, fruit and/or leaf of the plant) by physically removing a piece of such plant, such as grinding a flower of the plant. Such compounds may also be isolated from the plant by using extraction procedures well known in the art (e.g., the use of organic solvents such as lower C₁-C₈ alcohols, C₁-C₈ alkyl polyols, C₁-C₈ alkyl ketones, C₁-C₈ alkyl ethers, acetic acid C₁-C₈ alkyl esters, and chloroform, and/or inorganic solvents such as water, inorganic acids such as hydrochloric acid, and inorganic bases such as sodium hydroxide). In one embodiment, the Malva sylvestris extract contains only hydrophilic compounds (e.g., isolated by using a hydrophilic solvent, such as water or ethanol). In one embodiment, the Malva sylvestris extract is an aqueous extract from the flowers.

In one embodiment, the extract is present in the composition in an amount from about 0.001% to about 20% by weight, in particular in an amount from about 0.01% to about 10% by weight. Unless stated otherwise, the weight of the extract refers to the dry weight of the extract.

Cotinus Coggygria Extract

What is meant by a “Cotinus coggygria extract” is a blend of compounds isolated from a Cotinus coggygria plant. In one embodiment, the compounds are isolated from the leaf of the plant. In a further embodiment, the compounds are isolated from dried leaves of the plant. Such compounds may be isolated from one or more parts of the plant (e.g., the whole plant, flower, seed, root, rhizome, bark, wood, stem, fruit and/or leaf of the plant) by physically removing a piece of such plant, such as grinding a root of the plant. Such compounds may also be isolated from the plant by using extraction procedures well known in the art (e.g., the use of organic solvents such as lower C₁-C₈ alcohols, C₁-C₈ alkyl polyols, C₁-C₈ alkyl ketones, C₁-C₈ alkyl ethers, acetic acid C₁-C₈ alkyl esters, and chloroform, and/or inorganic solvents such as water, inorganic acids such as hydrochloric acid, and inorganic bases such as sodium hydroxide). In one embodiment, the Cotinus coggygria extract contains only hydrophilic compounds (e.g., isolated by using a hydrophilic solvent, such as water or ethanol). In one embodiment, the Cotinus coggygria extract is an aqueous extract from the leaf of Cotinus coggygria.

In one embodiment, the extract is present in the composition in an amount from about 0.001% to about 20% by weight, in particular in an amount from about 0.01% to about 10% by weight. Unless stated otherwise, the weight of the extract refers to the dry weight of the extract.

Other Extracts

In one embodiment, the compositions of the present invention contain one or more of the extracts from plants selected from the group consisting of matricaria chamomilla, thymus vulgaris, and matricaria recutita. In one embodiment, the extract is present in the composition in an amount from about 0.001% to about 20% by weight, in particular in an amount from about 0.01% to about 10% by weight. Unless stated otherwise, the weight of the extract refers to the dry weight of the extract.

Compositions

The compositions useful in the present invention involve formulations suitable for administering to the target tissues. In one embodiment, the composition contains a safe and effective amount of (i) Malva sylvestris extract and/or cotinus coggygria extract and (ii) a cosmetically-acceptable carrier. In one embodiment, the cosmetically-acceptable carrier is from about 50% to abut 99.99%, by weight, of the composition (e.g., from about 80% to about 99%, by weight, of the composition).

The compositions may be made into a wide variety of product types that include but are not limited to solutions, suspensions, lotions, creams, gels, sticks, sprays, ointments, cleansing liquid washes and solid bars, shampoos and hair conditioners, pastes, foams, powders, mousses, shaving creams, wipes, patches, nail lacquers, wound dressing and adhesive bandages, hydrogels, film-forming products, facial and skin masks, make-up such as foundations, mascaras, and lipsticks, liquid drops, vaginal washes, suppositories, tampons, toothpastes, mouthwashes, lozenges, tablets, gums and candy, mucoadhesives, and the like. These product types may contain several types of cosmetically-acceptable carriers including, but not limited to solutions, suspensions, emulsions such as microemulsions and nanoemulsions, gels, solids and liposomes. The following are non-limitative examples of such carriers. Other carriers can be formulated by those of ordinary skill in the art.

The compositions useful in the present invention can be formulated as solutions. Solutions typically include an aqueous or organic solvent (e.g., from about 50% to about 99.99% or from about 90% to about 99% of a cosmetically acceptable aqueous or organic solvent). Examples of suitable organic solvents include: propylene glycol, polyethylene glycol (200-600), polypropylene glycol (425-2025), glycerol, 1,2,4-butanetriol, sorbitol esters, 1,2,6-hexanetriol, ethanol, and mixtures thereof.

A lotion can be made from such a solution. Lotions typically contain from about 1% to about 20% (e.g., from about 5% to about 10%) of an emollient(s) and from about 50% to about 90% (e.g., from about 60% to about 80%) of water. As used herein, “emollients” refer to materials used for the prevention or relief of dryness, as well as for the protection of the skin or hair. Examples of emollients include, but are not limited to, those set forth in the International Cosmetic Ingredient Dictionary and Handbook, eds. Wenninger and McEwen, pp. 1656-61, 1626, and 1654-55 (The Cosmetic, Toiletry, and Fragrance Assoc., Washington, D.C., 7^{th} Edition, 1997) (hereinafter “ICI Handbook”).

Another type of product that may be formulated from a solution is a cream. A cream typically contains from about 5% to about 50% (e.g., from about 10% to about 20%) of an emollient(s) and from about 45% to about 85% (e.g., from about 50% to about 75%) of water.

Yet another type of product that may be formulated from a solution is an ointment. An ointment may contain a simple base of animal, vegetable, or synthetic oils or semi-solid hydrocarbons. An ointment may contain from about 2% to about 10% of an emollient(s) plus from about 0.1% to about 2% of a thickening agent(s). Examples of thickening agents include, but are not limited to, those set forth in the ICI Handbook pp. 1693-1697.

The compositions useful in the present invention can also be formulated as emulsions. If the carrier is an emulsion, from about 1% to about 10% (e.g., from about 2% to about 5%) of the carrier contains an emulsifier(s). Emulsifiers may be nonionic, anionic or cationic. Examples of emulsifiers include, but are not limited to, those set forth in the ICI Handbook, pp. 1673-1686.

Lotions and creams can be formulated as emulsions. Typically such lotions contain from 0.5% to about 5% of an emulsifier(s), while such creams would typically contain from about 1% to about 20% (e.g., from about 5% to about 10%) of an emollient(s); from about 20% to about 80% (e.g., from 30% to about 70%) of water; and from about 1% to about 10% (e.g., from about 2% to about 5%) of an emulsifier(s).

Single emulsion skin care preparations, such as lotions and creams, of the oil-in-water type and water-in-oil type are well-known in the art and are useful in the subject invention. Multiphase emulsion compositions, such as the water-in-oil-in-water type or the oil-in-water-in-oil type, are also useful in the subject invention. In general, such single or multiphase emulsions contain water, emollients, and emulsifiers as essential ingredients.

The compositions of this invention can also be formulated as a gel (e.g., an aqueous, alcohol, alcohol/water, or oil gel using a suitable gelling agent(s)). Suitable gelling agents for aqueous and/or alcoholic gels include, but are not limited to, natural gums, acrylic acid and acrylate polymers and copolymers, and cellulose derivatives (e.g., hydroxymethyl cellulose and hydroxypropyl cellulose). Suitable gelling agents for oils (such as mineral oil) include, but are not limited to, hydrogenated butylene/ethylene/styrene copolymer and hydrogenated ethylene/propylene/styrene copolymer. Such gels typically contains between about 0.1% and 5%, by weight, of such gelling agents.

The compositions of the present invention can also be formulated into a solid formulation (e.g., a wax-based stick, soap bar composition, powder, wipe containing powder, lozenge, suppository, candy, or gum).

The compositions useful in the subject invention may contain, in addition to the aforementioned components, a wide variety of additional oil-soluble materials and/or water-soluble materials conventionally used in compositions for use on skin and mucosal tissues at their art-established levels.

Additional Cosmetically Active Agents

In one embodiment, the composition further contains another cosmetically active agent in addition to the extracts. What is meant by a “cosmetically active agent” is a compound (e.g., a synthetic compound or a compound isolated from a natural source, or a natural extract containing a mixture of compounds) that has a cosmetic or therapeutic effect on the tissue, including, but not limiting to, lightening agents, darkening agents such as self-tanning agents, anti-acne agents, shine control agents, anti-microbial agents such as anti-yeast agents, anti-fungal, and anti-bacterial agents, anti-inflammatory agents, anti-parasite agents, external analgesics, sunscreens, photoprotectors, antioxidants, keratolytic agents, detergents/surfactants, moisturizers, nutrients, vitamins, energy enhancers, anti-perspiration agents, astringents, deodorants, hair removers, hair growth enhancing agents, hair growth delaying agents, firming agents, anti-callous agents, agents for skin conditioning, anti-cellulite agents, fluorides, teeth whitening agents, anti-plaque agents, and plaque-dissolving agents, and odor-control agents such as odor masking or pH-changing agents.

In one embodiment, the agent is selected from, but not limited to, the group consisting of hydroxy acids, benzoyl peroxide, D-panthenol, octyl methoxycinnimate, titanium dioxide, octyl salicylate, homosalate, avobenzone, carotenoids, free radical scavengers, spin traps, retinoids and retinoid precursors such as retinol and retinyl palmitate, ceramides, polyunsaturated fatty acids, essential fatty acids, enzymes, enzyme inhibitors, minerals, hormones such as estrogens, steroids such as hydrocortisone, 2-dimethylaminoethanol, copper salts such as copper chloride, peptides containing copper such as Cu:Gly-His-Lys, coenzyme Q10, amino acids such a proline, vitamins, lactobionic acid, acetyl-coenzyme A, niacin, riboflavin, thiamin, ribose, electron transporters such as NADH and FADH2, and other botanical extracts such as aloe vera, Feverfew, and Soy, and derivatives and mixtures thereof. The cosmetically active agent will typically be present in the composition of the invention in an amount of from about 0.001% to about 20% by weight of the composition, e.g., about 0.005% to about 10% such as about 0.01% to about 5%.

Examples of vitamins include, but are not limited to, vitamin A, vitamin Bs such as vitamin B3, vitamin B5, and vitamin B12, vitamin C, vitamin K, vitamin E such as alpha, gamma or delta-tocopherol, and derivatives and mixtures thereof.

Examples of hydroxy acids include, but are not limited, to glycolic acid, lactic acid, malic acid, salicylic acid, citric acid, and tartaric acid.

Examples of antioxidants include, but are not limited to, water-soluble antioxidants such as sulfhydryl compounds and their derivatives (e.g., sodium metabisulfite and N-acetyl-cysteine), lipoic acid and dihydrolipoic acid, resveratrol, lactoferrin, and ascorbic acid and ascorbic acid derivatives (e.g., ascorbyl palmitate and ascorbyl polypeptide). Oil-soluble antioxidants suitable for use in the compositions of this invention include, but are not limited to, butylated hydroxytoluene, retinoids (e.g., retinol and retinyl palmitate), different types of tocopherols (e.g., alpha-, gamma-, and delta-tocopherols and their esters such as acetate) and their mixtures, tocotrienols, and ubiquinone. Natural extracts containing antioxidants suitable for use in the compositions of this invention, include, but not limited to, extracts containing flavonoids, isoflavonoids, and their derivatives such as genistein and diadzein (e.g., such as Soy and Clover extracts, extracts containing resveratrol and the like. Examples of such natural extracts include grape seed, green tea, pine bark, and propolis.

Other Materials

Various other materials may also be present in the compositions useful in the subject invention. These include humectants, proteins and polypeptides, preservatives and an alkaline agent. Examples of such agents are disclosed in the ICI Handbook, pp. 1650-1667. The compositions of the present invention may also contain chelating agents (e.g., EDTA) and preservatives (e.g., parabens). Examples of suitable preservatives and chelating agents are listed in pp. 1626 and 1654-55 of the ICI Handbook. In addition, the compositions useful herein can contain conventional cosmetic adjuvants, such as colorants such as dyes and pigments, opacifiers (e.g., titanium dioxide), and fragrances.

Mineral Water

The compositions of the present invention may be prepared using a mineral water, for example mineral water that has been naturally mineralized such as Evian® Mineral Water (Evian, France). In one embodiment, the mineral water has a mineralization of at least about 200 mg/L (e.g., from about 300 mg/L to about 1000 mg/L). In one embodiment, the mineral water contains at least about 10 mg/L of calcium and/or at least about 5 mg/L of magnesium.

The composition and formulations containing such compositions of the present invention may be prepared using methodology that is well known by an artisan of ordinary skill.

### EXAMPLE 1

### Extract Preparations

The following is a description of the preparation of various extracts of the present invention. As used in the subsequent Examples, the weight percentage of extract refers to the weight of the liquid extract.

A: Malva Sylvestris Extract Preparation.

Malva sylvestris (whole dried flowers) was purchased from Botanic Choice (Hobart, Ind.) or Bilek (Troyan, Bulgaria). Ten grams of whole flowers were placed in 200 ml cold water, and brought to boiling in a sealed container. After the appearance of the boiling bubbles, the container was immediately withdrawn from the heating source, covered, and stored at room temperature for from about 1 hour to about 12 hours, with occasional agitation. The extract was then filtered through gauze, and excess liquid was squeezed manually from herbs to maximize the extract yield. The extract was further filtered through 22-micrometer 250 ml filtering unit from Nalgene (Rochester, N.Y.), under vacuum.

Alternatively, Malva extract can be prepared by adding ten grams of whole flowers to 200 ml cold water, and agitating the mixture at room temperature for from about 1 hour to about 12 hours.

B: Cotinus Coggygria Extract Preparation.

Cotinus coggygria herb (whole dried leaf) was purchased from Bilkokoop (Sofia, Bulgaria). Ten grams of whole leaves were placed in 100 ml cold water, and brought to boiling in a sealed container, and boiled for 5 minutes. The container was then immediately withdrawn from the heating source, covered, and stored at room temperature for from about 1 hour to about 12 hours, with occasional agitation. After this, the extract was filtered through gauze, and excess liquid was squeezed manually from herbs to maximize the extract yield. The extract was further filtered through 22-micrometer 250 ml filtering unit from Nalgene (Rochester, N.Y.), under vacuum.

C: Matricaria Chamomilla Extract Preparation

Matricaria chamomilla herb (whole dried flowers) was purchased from Bilek (Troyan, Bulgaria). Matricaria recutita herb (whole dried flowers) was purchased from Botanic Choice (Hobart, Ind.). Ten grams of whole flowers were placed in 200 ml cold water, and brought to boiling in a sealed container. After the appearance of the boiling bubbles, the container was immediately withdrawn from the heating source, covered, and stored at room temperature for from about 1 hour to about 12 hours, with occasional agitation. After this, the extract was filtered through gauze, and excess liquid was squeezed manually from herbs to maximize the extract yield The extract was further filtered through 22-micrometer 250 ml filtering unit from Nalgene (Rochester, N.Y.), under vacuum.

D: Arctostaphylos uva-ursi Extract Preparation.

Arctostaphylos uva-ursi herb (whole dried leaf) was purchased from Bilkokoop (Sofia, Bulgaria). Ten grams of whole leaves were placed in 100 ml cold water, and brought to boiling in a sealed container, and boiled for 5 minutes. The container was then immediately withdrawn from the heating source, covered, and stored at room temperature for from about 1 hour to about 12 hours, with occasional agitation. After this, the extract was filtered through gauze, and excess liquid was squeezed manually from herbs to maximize the extract yield. The extract was further filtered through 22-micrometer 250 ml filtering unit from Nalgene (Rochester, N.Y.), under vacuum.

E: Herbal Combination Extract Preparation

Malva sylvestris herb (whole dried flowers) was purchased from both Bilek (Troyan, Bulgaria) or Botanic Choice (Hobart, Ind.). Matricaria chamomilla herb (whole dried flowers) was purchased from Bilek (Troyan, Bulgaria). Matricaria recutita was purchased from Botanic Choice (Hobart, Ind.). Thymus vulgaris herb (dried stem) was purchased from Bilek (Troyan, Bulgaria). Amounts of herbs, as described in Tables 1 and 2 below, were placed together in 200 ml cold water and brought to boiling in a sealed container. After the appearance of the boiling bubbles, the container was immediately withdrawn from the heating source, covered, and stored at room temperature for from about 1 hour to about 12 hours with occasional agitation. The extract was then filtered through gauze, and excess liquid was squeezed manually from herbs to maximize the extract yield. The extract was further filtered through 22-micrometer 250 ml filtering unit from Nalgene (Rochester, N.Y.), under vacuum.

TABLE 1

| Name | Amount | |
|---|---|---|
| Malva sylvestris L. | 2 | g |
| Thymus vulgaris | 4 | g |
| Matricaria chamomilla L. | 4 | g |
| Water | 200 | ml |

TABLE 2

| Name | Amount | |
|---|---|---|
| Malva sylvestris L. | 2 | g |
| Thymus vulgaris | 4 | g |
| Matricaria recutita L. | 4 | g |
| Water | 200 | ml |

### EXAMPLE 2

### Enhancement of Elastin Promoter Activity

Rat cardiac myoblasts H₉C2 were purchased from ATCC (Manassas, Va.). Cultures were maintained in Dulbecco's modified Eagle's medium (DMEM, Invitrogen Life Technologies, Carlsbad, Calif.) supplemented with 10% fetal bovine serum, 2 mM glutamine, 100 units/ml penicillin, and 50 μg/ml streptomycin (Invitrogen life technologies, Carlsbad, Calif.).

Cell cultures were transiently transfected with the elastin promoter-luciferase reporter construct (Elp2.2, a 2.2 kb elastin promoter fragment from nt −2267 to nt +2, driving the firefly luciferase gene, which was obtained from Promega, Madison Wis.). DNA was prepared by Qiagen Maxi columns (Qiagen Valencia, Calif.). In all transfections, a construct with the thymidine kinase promoter and the Renilla luciferase reporter gene (pRL-TK, Promega, Madison Wis.) was included as an internal control. Cells were plated at 4×10⁴ in each well of a 24-well plate (Corning Incorporated, Corning, N.Y.) in growth media without antibiotics for 24 hours, reaching 80-90% confluency at the time of transfection. Typically, cells were transfected with 0.8 μg DNA per well using Lipofectamine 2000 (Invitrogen life technologies, Carlsbad, Calif.). One day after transfection, cells were treated with agents at indicated concentrations for approximately 48 hours before they were lysed for luciferase assays, using Dual-Luciferase Reporter System from Promega (Madison, Wis.), following manufacture's protocol. Briefly, the firefly luciferase activity was measured first (representing elastin promoter activity), followed by the renilla luciferase (internal control), using luminometer LMAX, from Molecular Devices (Sunnyvale, Calif.). The ratio of these two luciferase activities (RLU) was used to evaluate the activity of each promoter.

Cells were treated with various doses of Malva Sylvestris extract (Example 1A), Coggygria extract (Example 1B), Matricaria chomomilla extract (Example 1C), Arctostaphylos uva-ursi extract (Example 1D), M. sylvestris/M. chamomilla/Thymus vulgaris extract (Example 1E), or M. sylvestris/M. recutita/Thymus vulgaris extract (Example 1C) and the effect of the extract on the induction of expression from the elastin promoter was evaluated. The extracts were added to the transfected H₉C2 cells and were incubated for 48 hours. An increase in elastin promoter activity was observed in the presence of increasing doses of the extracts, as compared to untreated cells, as shown in Table 3. This example demonstrates that each of the extracts could enhance elastin production.

TABLE 3

| Agent (% W/W) | Induction |
|---|---|
| Control - no extract added | 1 +/− 0 |
| Malva sylvestris (2.5%) | 1.93 +/− 0.33 |
| Malva sylvestris (5%) | 2.27 +/− 0.03 |
| Cotinus coggygria (0.05%) | 1.75 +/− 0.52 |
| Cotinus coggygria (0.1%) | 1.62 +/− 0.3 |
| Cotinus coggygria (0.15%) | 1.5 +/− 0 |
| Matricaria chamomilla (5%) | 1.65 +/− 0.25 |
| Arctostaphylos uva-ursi (2.5%) | 1.56 +/− 0.34 |
| Malva sylvestris (5%) and Cotinus | 2.7 +/− 0 |
| coggygria (0.1%) | |
| Malva sylvestris (2.5%) and | 2.9 +/− 0.56 |
| Arctostaphylos uva-ursi (2.5%) | |
| Cotinus coggygria (0.05%)and | 2.27 +/− 0 |
| Arctostaphylos uva-ursi (2.5%) | |
| Malva sylvestris/Matricaria | 1.66 +/− 0 |
| recutita/Thymus vulgaris (2%) | |
| Malva sylvestris/Matricaria | 2.2 +/− 0 |
| chamomilla/Thymus vulgaris (2%) | |
| Malva sylvestris/Matricaria | 3.3 +/− 0 |
| chamomilla/Th. Vulgaris (2%) and Cotinus | |
| coggygria (0.15%) | |

### EXAMPLE 3

### Protection from Elastase Degradation

Human leukocyte elastase (HLE) was purchased from Sigma (St. Louis, Mo.), and reconstituted at 1 unit/ml in phosphate buffered saline (PBS, Invitrogen life Technologies, Carlsbad, Calif.). Soluble bovine neck ligament elastin labeled with BODIPY FL dye was purchased from Molecular Probes, Inc. (Eugene, Oreg.), such that the fluorescence was quenched in the conjugate, and could be activated upon elastase digestion. Human leukocyte elastase (0.0625 U/ml), elastin substrate (25 μg/ml), and increasing concentrations of test material were incubated for one hour at room temperature. Fluorescence was measured at excitation at 490 nm and emission at 520 nm using a fluorescent plate reader Gemini from Molecular Devices (Sunnyvale, Calif.). Background fluorescence of substrate alone had been subtracted from each measurement.

Two batches of Cotinus coggygria extracts, prepared according to Example 1B, were averaged in the experiment, with data presented as compared to controls with no extract added. Cotinus coggygria extracts inhibited HLE activity in a dose dependent manner as shown in Table 4. As low as 0.01% of Cotinus coggygria extract resulted in approximately 60% reduction in HLE activity, while 0.1% of extract almost completely inhibited elastase activity. This example demonstrates that Cotinus extract can protect elastin fibers from damage and degradation.

TABLE 4

| Cotinus Extract (% W/W) | Elastase Inhibition (%) |
|---|---|
| 0 | 0 +/− 1.6 |
| 0.0001 | 2.8 +/− 1.2 |
| 0.001 | 15.35 +/− 5.85 |
| 0.01 | 50 +/− 15 |
| 0.1 | 97.6 +/− 0 |

### EXAMPLE 4

### Protection from Trypsin Degradation

Trypsin was purchased from Sigma (St. Louis, Mo.), and reconstituted at 2000 unit/ml in phosphate buffered saline (PBS, Invitrogen life technologies, Carlsbad, Calif.). Casein labeled with BODIPY FL dye was purchased from Molecular Probes, Inc., (Eugene, Oreg.), such that the fluorescence was quenched in the conjugate, and could be activated upon protease digestion. Trypsin (500 U/ml), Casein (10 μg/ml), and increasing concentrations of test agent, were incubated for two hours at room temperature. Fluorescence was measured at excitation at 485 nm and emission at 538 nm using a fluorescent plate reader Gemini from Molecular Devices (Sunnyvale, Calif.). Background fluorescence of substrate alone had been subtracted from each measurement.

Two batches of Cotinus coggygria extracts, prepared as described in Example 1B, were averaged in the experiment, with data presented as compared to controls with no extract added. Cotinus coggygria extract inhibited trypsin activity in a dose dependent manner as shown in Table 5. As low as 0.02% of Cotinus coggygria extract resulted in approximately 35% reduction in trypsin activity, while addition of 0.1% of extract resulted in approximately 61% inhibition of trypsin activity. This example demonstrates that Cotinus extract can protect tissues from proteolytic damage and degradation, therefore maintaining tissue integrity.

TABLE 5

| Cotinus Extract (% W/W) | Trypsin Inhibition (%) |
|---|---|
| 0 | 0 +/− 0 |
| 0.008 | 0 +/− 0 |
| 0.004 | 6.4 +/− 6.4 |
| 0.02 | 34.6 +/− 14.9 |
| 0.1 | 60.9 +/− 6.2 |

It is understood that while the invention has been described in conjunction with the detailed description thereof, that the foregoing description is intended to illustrate and not limit the scope of the invention, which is defined by the scope of the appended claims. Other aspects, advantages, and modifications are within the claims.

### EXAMPLE 5

### Protection from Matrix Metalloproteinase Degradation

Human macrophage elastase (HME, also named Matrix Metalloproteinase-12, MMP-12) and fluorescently labeled substrate were purchased from R&D systems (Minneapolis, Minn.). The fluorescence was quenched in the substrate, and could be activated upon elastase digestion. HME (100 ng/ml), substrate (10 μg/ml), and increasing concentrations of test material were incubated for one hour at room temperature. Fluorescence was measured at excitation at 320 nm and emission at 405 nm using a fluorescent plate reader Gemini from Molecular Devices (Sunnyvale, Calif.). Background fluorescence of substrate alone had been subtracted from each measurement.

Two batches of Cotinus coggygria extracts, prepared according to Example 1B, were averaged in the experiment, with data presented as compared to controls with no extract added. Cotinus coggygria extracts inhibited HME activity in a dose dependent manner as shown in Table 6. As low as 0.01% of Cotinus coggygria extract resulted in approximately 40% reduction in HME activity, while 0.5% of extract almost completely inhibited HME activity. This example demonstrates that Cotinus extract can protect elastin fibers from damage and degradation.

TABLE 6

| Cotinus Extract (% W/W) | HME Inhibition (%) |
|---|---|
| 0 | 0 |
| 0.01 | 37.6 +/− 2.4 |
| 0.05 | 69.6 +/− 1.0 |
| 0.1 | 79.5 +/− 1.5 |
| 0.5 | 96.3 +/− 0.4 |

Malva extracts, prepared according to Example 1A, were tested in the experiment, with data presented as compared to controls with no extract added. Malva extract inhibited HME activity in a dose dependent manner as shown in Table 7. As low as 0.6% of Malva extract resulted in approximately 23% reduction in HME activity, while 5% of extract inhibited HME activity 80%. This example demonstrates that Malva extract can protect elastin fibers from damage and degradation.

TABLE 7

| Malva Extract (% W/W) | HME Inhibition (%) |
|---|---|
| 0 | 0 |
| 0.6 | 22.0 +/− 0.9 |
| 1.25 | 40.1 +/− 0.0 |
| 2.5 | 62.0 +/− 0.6 |
| 5 | 79.3 +/− 1.2 |

Arctostaphylos uva-ursi extracts, prepared according to Example 1D, were tested in the experiment, with data presented as compared to controls with no extract added. Arctostaphylos uva-ursi extract inhibited HME activity in a dose dependent manner as shown in Table 8. As low as 0.01% of Arctostaphylos uva-ursi extract resulted in approximately 10% reduction in HME activity, while 0.5% of extract inhibited HME activity 90%. This example demonstrates that Arctostaphylos uva-ursi extract can protect elastin fibers from damage and degradation.

TABLE 8

| Arctostaphylos uva-ursi | |
|---|---|
| Extract (% W/W) | HME Inhibition (%) |
| 0 | 0 |
| 0.01 | 10.8 +/− 2.0 |
| 0.05 | 44.9 +/− 0.4 |
| 0.1 | 62.4 +/− 1.8 |
| 0.5 | 89.5 +/− 0.5 |

What is claimed is:

1. A method of enhancing the elasticity or structural integrity of skin or mucosal tissue, said method comprising administering to skin or mucosal tissue in need of such enhancement a composition comprising a safe and effective amount of Malva sylvestris extract.

2. A method of claim 1, wherein said method comprises enhancing the elasticity or structural integrity of the skin.

3. A method of claim 1, wherein said method comprises enhancing the elasticity or structural integrity of vaginal mucosal tissue.

4. A method of claim 1, wherein said method comprises enhancing the elasticity or structural integrity and function of oral mucosal tissue.

5. A method of claim 1, wherein said composition comprises from about 0.1%, by weight, to about 20%, by weight, of said Malva sylvestris extract.

6. A method of claim 2, wherein said composition comprises from about 0.1%, by weight, to about 20%, by weight, of said Malva sylvestris extract.

7. A method of claim 3, wherein said composition comprises from about 0.1%, by weight, to about 20%, by weight, of said Malva sylvestris extract.

8. A method of claim 4, wherein said composition comprises from about 0.1%, by weight, to about 20%, by weight, of said Malva sylvestris extract.

9. A product comprising:
(a) a composition comprising a Malva sylvestris extract; and
(b) instructions directing the user to apply said composition to skin or mucosal tissue in order to enhance the elasticity or structural integrity of said skin or mucosal tissue.

10. A product of claim 9, wherein said instructions direct the user to apply said composition to the skin or mucosal tissue in order to enhance the elasticity or structural integrity of skin.

11. A product of claim 9, wherein said instructions direct the user to apply said composition to the skin or mucosal tissue in order to enhance the elasticity or structural integrity of vaginal mucosal tissue.

12. A product of claim 9, wherein said instructions direct the user to apply said composition to the skin or mucosal tissue in order to enhance the elasticity or structural integrity of oral mucosal tissue.

13. A product of claim 9, wherein said composition comprises from about 0.1%, by weight, to about 20%, by weight, of said Malva sylvestris extract.

14. A product of claim 10, wherein said composition comprises from about 0.1%, by weight, to about 20%, by weight, of said Malva sylvestris extract.

15. A product of claim 11, wherein said composition comprises from about 0.1%, by weight, to about 20%, by weight, of said Malva sylvestris extract.

16. A product of claim 12, wherein said composition comprises from about 0.1%, by weight, to about 20%, by weight, of said Malva sylvestris extract.

17. A method of promoting a product comprising a composition comprising a Malva sylvestris extract, wherein said method comprises directing the user to apply said composition to skin or mucosal tissue to enhance the elasticity or structure integrity of said skin or mucosal tissue.

18. A method of claim 17, wherein said method comprises directing the user to apply said composition to skin or mucosal tissue to enhance the elasticity or structure integrity of skin.

19. A method of claim 17, wherein said method comprises directing the user to apply said composition to skin or mucosal tissue to enhance the elasticity or structure integrity of vaginal mucosal tissue.

20. A method of claim 17, wherein said method comprises directing the user to apply said composition to skin or mucosal tissue to enhance the elasticity or structure integrity of oral mucosal tissue.

21. A method of claim 17, wherein said composition comprises from about 0.1%, by weight, to about 20%, by weight, of said Malva sylvestris extract.

22. A method of claim 18, wherein said composition comprises from about 0.1%, by weight, to about 20%, by weight, of said Malva sylvestris extract.

23. A method of claim 19, wherein said composition comprises from about 0.1%, by weight, to about 20%, by weight, of said Malva sylvestris extract.

24. A method of claim 20, wherein said composition comprises from about 0.1%, by weight, to about 20%, by weight, of said Malva sylvestris extract.
